(19) United States
(12) Patent Application Publication
(10) Pub. No.: US 2026/0368354 A1
(43) Pub. Date: Nov. 19, 2026

(54) TERMINAL APPARATUS, INFORMATION PROCESSING PROGRAM, AND INFORMATION PROCESSING SYSTEM

(71) Applicant: SONY GROUP CORPORATION, TOKYO (JP)

(72) Inventors: JUNICHI KOSAKA, TOKYO (JP); MASAHARU ASANO, TOKYO (JP)

(21) Appl. No.: 19/154,488

(22) PCT Filed: Jan. 11, 2024
(86) PCT No.: PCT/JP2024/000340

§ 371 Date: Aug. 7, 2025

(30) Foreign Application Priority Data
Feb. 28, 2023 (JP) 2023-030531

(51) Int. Cl. G06F 21/62

(52) U.S. Cl. CPC G06F 21/6245

(57) ABSTRACT

A terminal apparatus includes a disclosure approval section that receives, from a service server, a request for an approval for disclosure of data regarding a first user, the data being accumulated in a database, and that accepts the disclosure approval input by a second user who is different from the first user; a provision processing section that performs processing to provide the service server with the data, which is a target for the disclosure approval; and an approval information recording section that records disclosure approval information regarding the disclosure approval in a history recording system in a network.

### TECHNICAL FIELD

The present disclosure relates to a terminal apparatus that approves for disclosure of data, an information processing program that is executed by the terminal apparatus, and an information processing system that includes the terminal apparatus and a service server that provides services that use disclosed data.

### BACKGROUND ART

Instead of the centralized Internet (what is called Web 2.0), the decentralized Internet (what is called Web 3.0) has been sought. With Web 3.0, data ownership is distributed. This enables personal data to be managed by an individual who is its original owner.

### CITATION LIST

### Patent Literature

Patent Literature 1: Japanese Patent Application Laid-open No. 2011-221966

### DISCLOSURE OF INVENTION

### Technical Problem

With Web 2.0, there is a need to register user information in order to use services. In this case, conditions for disclosing data of minors can be set by setting parental control with respect to, for example, handling of the data of minors. For example, Patent Literature 1 discloses a technology used to appropriately restrict use of electronic content stored in a recording medium, according to an attribute of an entity that uses the electronic content.

On the other hand, individuals are left to manage their own user data in an environment of Web 3.0. Users who do not have sufficient ability to perform determination (such as minors and elderly people) are discussed. Data is recorded in devices of such users, but it is desirable that the data be managed by determination being performed by supervisors (such as parents and guardians) of the users.

### Solution to Problem

A terminal apparatus according to an embodiment of the present disclosure includes a disclosure approval section that receives, from a service server, a request for an approval for disclosure of data regarding a first user, the data being accumulated in a database, and that accepts the disclosure approval input by a second user who is different from the first user; a provision processing section that performs processing to provide the service server with the data, which is a target for the disclosure approval; and an approval information recording section that records disclosure approval information regarding the disclosure approval in a history recording system in a network.

With Web 3.0, there is essentially no need to register user information upon using services, which is different from Web 2.0. Thus, it is understood that, when there is a need to disclose user data of the first user, it is necessary that the second user responsible for the first user perform management determination.

The disclosure approval information may include date-and-time information, information used to identify the first user, information used to identify the second user, and information used to identify the service server.

There may be a plurality of second users or there may be a change in second user. When it is necessary that the second user perform management determination successively, an importance of recording information regarding when and who determined that a disclosure approval for which user data is to be given is especially increased in such a case.

The disclosure approval section may individually approve disclosure of the pieces of data of different types, and the disclosure approval information may further include information indicating the type of the piece of data.

Examples of the service for which there is a need to disclose user data include an online-examination service. It is conceivable that, upon using an online-examination service, first user's pieces of vital data of different types (such as a body temperature, a heart rate, a breathing rate, and a blood pressure) could be provided to the service server in order to get medical advice. In such a case, information regarding when and who determined that a disclosure approval for which piece of vital data is to be given can be recorded.

The disclosure approval section may specify a purpose for which the disclosure-approval-target data is allowed to be disclosed, and the disclosure approval information may further include information indicating the purpose.

The present embodiment enables the second user to determine a disclosure-allowable purpose for each type of vital data. Information regarding when and who determined that a disclosure approval for which vital data and for which purpose is to be given can be recorded.

The disclosure approval section may specify a period of time for which the disclosure-approval-target data is allowed to be disclosed, and the disclosure approval information may further include information indicating the period of time.

The present embodiment enables the second user to determine a disclosure-allowable period of time for each type of vital data. Information regarding when and who determined that a disclosure approval for which vital data and for which period of time is to be given can be recorded.

The disclosure approval section may specify a user to which the disclosure-approval-target data is allowed to be disclosed, and the disclosure approval information may further include information indicating the user.

The present embodiment enables the second user to determine a disclosure-allowable user for each type of vital data. Information regarding when and who determined that a disclosure approval for which vital data and for which user is to be given can be recorded.

The provision processing section may acquire the disclosure-approval-target data from the database, and may supply the acquired data to the service server.

The provision processing section performs processing to provide the service server with disclosure-approval-target vital data. For example, it is sufficient if the provision processing section acquires a piece of disclosure-approval-target vital data from the database (a wearable device, a mobile device of the first user, or at least one storage apparatus in a network) in which pieces of vital data are accumulated, and supplies the piece of disclosure-approval-target vital data to the service server. This results in synchronizing vital data in the database with vital data in the service server.

The provision processing section may report the target for the disclosure approval to the service server, and may cause the service server to acquire the disclosure-approval-target data from the database.

The provision processing section performs processing to provide the service server with disclosure-approval-target vital data. For example, the provision processing section may report a disclosure-approval target to the service server, and may cause the service server to acquire vital data of the disclosure-approval target from the database (a wearable device, a mobile device of the first user, or at least one storage apparatus in a network). This results in synchronizing vital data in the database with vital data in the service server.

The database may be provided to at least one of a wearable device worn by the first user; a mobile device that communicates with the wearable device; the terminal apparatus that communicates with the wearable device or the mobile device; or at least one storage apparatus, in a network, that communicates with the wearable device, the mobile device, or the terminal apparatus.

The database may be a nonvolatile storage apparatus built in the wearable device, or may be a nonvolatile storage apparatus provided to a device that can receive data from the wearable device. It is sufficient if the at least one storage apparatus in the network is, for example, a server or a blockchain.

The history recording system in the network may be a blockchain.

It is difficult to falsify data recorded on a blockchain. In particular, recording of the disclosure approval information on a blockchain that serves as the history recording system makes it possible to record, in the form of tamper-proof traces, information indicating when and who determined that a disclosure approval for which vital data of whom is to be given.

The data regarding the first user may be vital data of the first user.

Examples of the service for which there is a need to disclose user data include an online-examination service. It is conceivable that, upon using an online-examination service, vital data (such as a body temperature, a heart rate, a breathing rate, and a blood pressure) acquired using a wearable device worn by the first user could be provided to the service server in order to get medical advice. In such a case, the second user (such as a parent or a guardian) different from the first user (such as a minor or an elderly person) can give a disclosure approval at a timing at which it is necessary to disclose vital data of the first user.

The second user may be a supervisor of the first user.

Individuals are left to manage their own user data in an environment of Web 3.0. Users who do not have sufficient ability to perform determination (such as minors and elderly people) are discussed. Data is recorded in devices of such users, but it is desirable that the data be managed by determination being performed by supervisors (such as parents and guardians) of the users.

The service server may record the second user or a plurality of the second users in association with the first user; and when the service server records the plurality of the second users, the service server may record the plurality of the second users in association with priorities, and may transmit the request for the disclosure approval, the request being made to the second user selected according to an order of priority.

The term “according to an order of priority” means that, for example, a request is transmitted to a highest-priority supervisor and that the request is transmitted to a second-highest-priority supervisor when there is no response from the highest-priority supervisor. When supervisors are prioritized without being treated in the same order, this makes it possible to give a higher priority to, for example, a supervisor who replies quickly, or a supervisor who knows a supervisee better. This results in being able to disclose vital data promptly and thus to perform an appropriate examination earlier.

When the service server records a plurality of the second users, the service server may record the plurality of the second users in association with priorities for each of the pieces of data of different types, and may transmit the request for the disclosure approval, the request being made to the second user selected according to an order of priority set for the type of the piece of disclosure-approval-target data; the disclosure approval section may individually approve disclosure of the pieces of data of the different types; and the disclosure approval information may further include information indicating the type of the piece of data.

When supervisors are prioritized for each of pieces of vital data of different types without being treated in the same order with respect to the pieces of vital data of different types, this makes it possible to give a higher priority to a supervisor who knows a supervisee's piece of vital data of a specific type better. For example, a mother is given a higher priority for a heart rate, and a father is given a higher priority for a body temperature. This results in being able to disclose pieces of vital data of different types appropriately and thus to perform an appropriate examination.

An information processing program according to an embodiment of the present disclosure causes a control circuit of a terminal apparatus to operate as a disclosure approval section, a provision processing section, and an approval information recording section, the disclosure approval section receiving, from a service server, a request for an approval for disclosure of data regarding a first user, the data being accumulated in a database, the disclosure approval section accepting the disclosure approval input by a second user who is different from the first user, the provision processing section performing processing to provide the service server with the data, which is a target for the disclosure approval, the approval information recording section recording disclosure approval information regarding the disclosure approval in a history recording system in a network.

An information processing system according to an embodiment of the present disclosure includes a service server, and a terminal apparatus including a disclosure approval section that receives, from the service server, a request for an approval for disclosure of data regarding a first user, the data being accumulated in a database, and that accepts the disclosure approval input by a second user who is different from the first user; a provision processing section that performs processing to provide the service server with the data, which is a target for the disclosure approval; and an approval information recording section that records disclosure approval information regarding the disclosure approval in a history recording system in a network.

### BRIEF DESCRIPTION OF DRAWINGS

FIG. 1 illustrates an information processing system according to a first embodiment of the present disclosure.

FIG. 2 illustrates a functional configuration of the information processing system.

FIG. 3 illustrates a flow of an operation performed by a service server.

FIG. 4 illustrates a flow of an operation performed by a supervisor terminal apparatus.

FIG. 5 illustrates an example of a disclosure approval screen according to a second embodiment.

FIG. 6 illustrates an example of the disclosure approval screen according to a third embodiment.

FIG. 7 illustrates an example of the disclosure approval screen according to a fourth embodiment.

### MODE(S) FOR CARRYING OUT THE INVENTION

### I. First Embodiment

### 1. Overview

Individuals are left to manage their own user data in an environment of Web 3.0. Users who do not have sufficient ability to perform determination (such as minors and elderly people) are discussed. Data is recorded in devices of such users, but it is desirable that the data be managed by determination being performed by supervisors (such as parents and guardians) of the users.

With Web 3.0, there is essentially no need to register user information upon using services, which is different from Web 2.0. Thus, it is understood that, when there is a need to disclose user data, timings at which management determination is performed are successively necessary. There may be a plurality of supervisors (such as a mother and a father) or there may be a change in supervisor (such as a change in a lawyer serving as a guardian). When it is necessary that supervisors perform management determination successively, an importance of recording information regarding when and who determined that a disclosure approval for which user data is to be given is especially increased in such a case.

Examples of the service for which there is a need to disclose user data include an online-examination service. It is conceivable that, upon using an online-examination service, vital data (such as a body temperature, a heart rate, a breathing rate, and a blood pressure) acquired using a wearable device worn by a user could be provided to a service server in order to get medical advice. In such a case, it is desirable that supervisors (such as parents or guardians) of, for example, minors or elderly people give a disclosure approval at a timing at which it is necessary to disclose vital data of, for example, the minors or the elderly people.

Note that it is understood that the present embodiment has advantages especially in the environment of Web 3.0, but is also applicable in an environment of Web 2.0.

Embodiments according to the present disclosure will now be described below with reference to the drawings.

### 2. Overview of Information Processing System

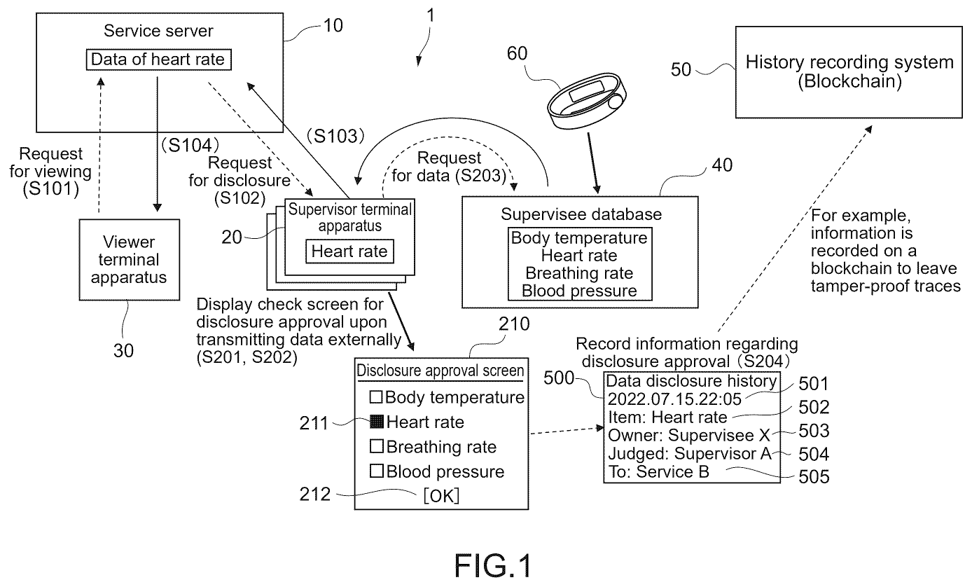

FIG. 1 illustrates an information processing system according to a first embodiment of the present disclosure.

An information processing system 1 includes a service server 10, a supervisor terminal apparatus 20 (a terminal apparatus), a viewer terminal apparatus 30, a supervisee database 40, and a history recording system 50 that are communicably connected to each other through a network such as the Internet.

The supervisee database 40 stores therein user data regarding a supervisee (a first user). In the present embodiment, the supervisee is a person such as a minor or elderly person who does not have sufficient ability to perform determination. In the present embodiment, the user data is vital data (such as a body temperature, a heart rate, a breathing rate, and a blood pressure). The vital data is data acquired using a wearable device 60 (such as a watch or wristband device) worn by a supervisee.

The supervisee database 40 may be a nonvolatile storage apparatus built in the wearable device 60, or may be a nonvolatile storage apparatus provided to a device that can receive data from the wearable device 60. In the latter case, the supervisee database 40 may be provided to, for example, a mobile device (that is not illustrated and is, for example, a smartphone used by a supervisee) that can communicate with the wearable device 60 through a network or using near field communication such as Bluetooth (registered trademark). Alternatively, the supervisee database 40 may be provided to the supervisor terminal apparatus 20 being capable of communicating with this mobile device and corresponding to a terminal apparatus (such as a smartphone) used by a supervisor who supervises a supervisee. Alternatively, the supervisee database 40 may be provided to at least one storage apparatus in a network. In this case, the at least one storage apparatus may acquire vital data from the wearable device 60, a terminal apparatus used by a supervisee, or the supervisor terminal apparatus 20. It is sufficient if the at least one storage apparatus in the network is, for example, a server or a blockchain.

The viewer terminal apparatus 30 is used by a viewer. The viewer is a person who views user data of a supervisee. In the present embodiment, the viewer is, for example, a doctor who views vital data of a supervisee who is a patient, in order to perform online examination. Examples of the viewer terminal apparatus 30 include a personal computer, a tablet computer, and a smartphone.

The supervisor terminal apparatus 20 is used by a supervisor (a second user). In the present embodiment, the supervisor is a person who supervises a supervisee and who is different from the supervisee. Examples of the supervisor of a minor may include a mother and a father. Examples of the supervisor of an elderly person may include a guardian such as a lawyer. Examples of the supervisor terminal apparatus 20 include a smartphone, a tablet computer, and a personal computer. When the viewer wants to view vital data of a supervisee, the supervisor gives a disclosure approval using a disclosure approval screen 210 displayed on the supervisor terminal apparatus 20.

The history recording system 50 is a recording system in a network, and records therein disclosure approval information 500 regarding a disclosure approval given by a supervisor through the supervisor terminal apparatus 20. The history recording system 50 may be a blockchain, or may also be an undistributed storage apparatus (such as a server).

The service server 10 is a web server that provides an interactive web application to the supervisor terminal apparatus 20 and the viewer terminal apparatus 30. In this example, the service server 10 provides a web service used when a viewer (a doctor) examines a patient (a supervisee). The service server 10 accepts a request indicating that a viewer (a doctor) who uses the viewer terminal apparatus 30 wants to view vital data of a patient (a supervisee) in order to perform examination. When the service server 10 acquires a disclosure approval from a viewer through the supervisor terminal apparatus 20, the service server 10 acquires vital data recorded in the supervisee database 40 and causes the vital data to be viewed by the viewer (the doctor) through the viewer terminal apparatus 30.

The example described above is an example of the use case according to the present embodiment. In other words, The attributes corresponding to a supervisor, a supervisee, and a viewer, the vital data corresponding to user data, and the examination web service corresponding to an interactive web application are merely examples, and the use case is not limited thereto. The examination web service may typically provide a remote online-examination service. However, the examination web service may provide a vital data disclosing service during face-to-face examination in medical facilities.

### 3. Functional Configuration of Information Processing System

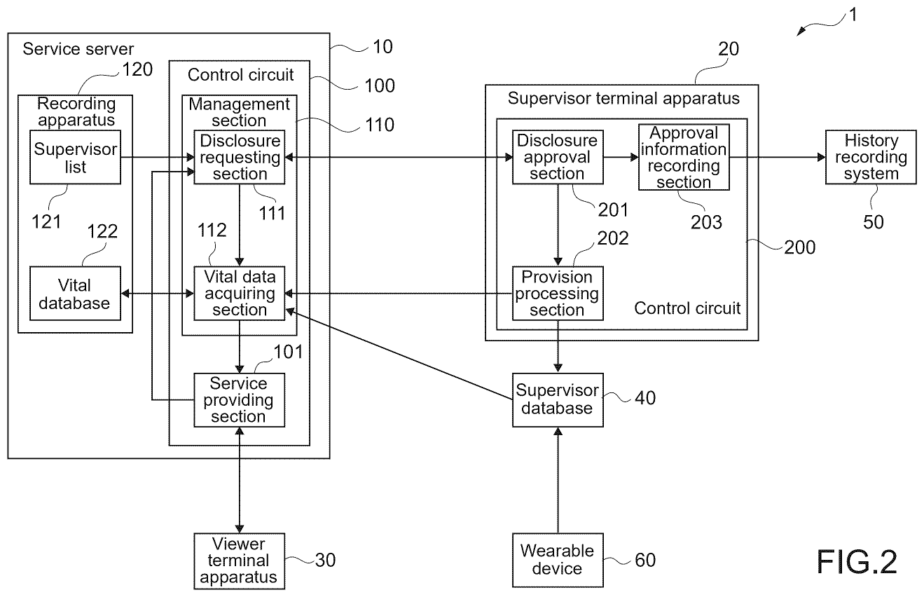

FIG. 2 illustrates a functional configuration of the information processing system.

The service server 10 operates as a service providing section 101 and a management section 110 by a CPU of a control circuit 100 loading, into a RAM, an information processing program recorded in a ROM and executing the program. The management section 110 includes a disclosure requesting section 111 and a vital data acquiring section 112. The service server 10 records a supervisor list 121 and a vital database 122 in a large-capacity nonvolatile recording apparatus 120 such as an HDD or an SSD. Note that the service providing section 101 and the management section 110 may be implemented by a physically different independent information processing apparatus (not illustrated).

The supervisor terminal apparatus 20 operates as a disclosure approval section 201, a provision processing section 202, and an approval information recording section 203 by a CPU of a control circuit 200 loading, into a RAM, an information processing program recorded in a ROM and executing the program.

### 4. Flow of Operation Performed by Information Processing System

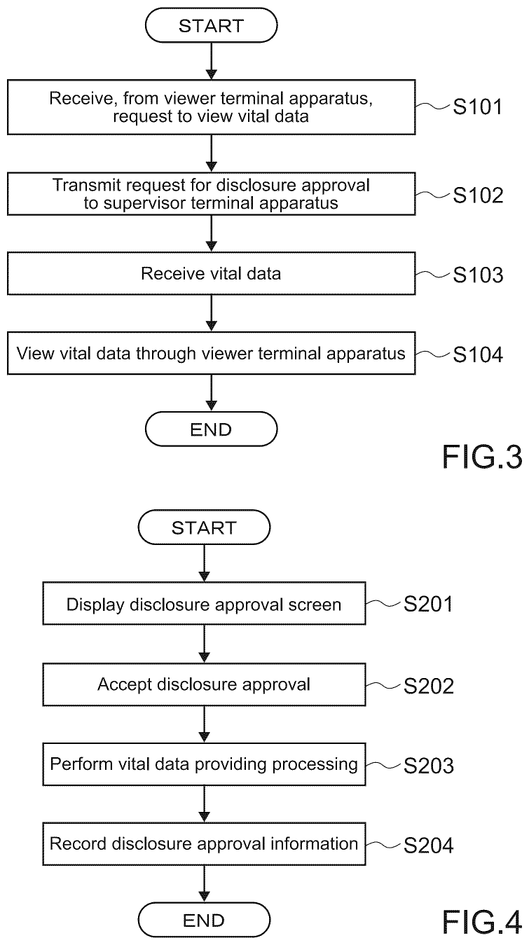

FIG. 3 illustrates a flow of an operation performed by the service server.

As a precondition, a supervisor accesses the service server 10 through the supervisor terminal apparatus 20 in order to cause a supervisee to be examined. A viewer (a doctor) accesses the service server 10 through the viewer terminal apparatus 30 in order to examine the supervisee. Respectively using the supervisor terminal apparatus 20 and the viewer terminal apparatus 30, the supervisor and the viewer (the doctor) communicate with each other (using, for example, a video call, a voice call, or a chat) on a web application provided by the service server 10. The viewer (the doctor) wants to view vital data of the supervisee during examination. This is the precondition for the operation flow.

In response to receiving input performed by the viewer (the doctor), the viewer terminal apparatus 30 transmits, to the service server 10, a request for an approval for disclosure of vital data regarding an examination-target supervisee.

The service providing section 101 of the service server 10 receives, from the viewer terminal apparatus 30, the request for an approval for disclosure of vital data regarding the examination-target supervisee (Step S101).

Referring to the supervisor list 121, the disclosure requesting section 111 of the service server 10 reads a supervisor recorded in association with a supervisee for the request for the disclosure approval. The supervisor list 121 records therein at least one supervisor in association with a supervisee. When a plurality of supervisors is recorded for a supervisee, the supervisor list 121 records therein the plurality of supervisors in association with priorities. For example, the supervisor list 121 records therein a plurality of supervisors (a mother and a father) for a supervisee (a minor), and records therein the plurality of supervisors in association with priorities (the mother =Prio 1, the father =Prio 2). The disclosure requesting section 111 transmits, to a supervisor recorded in the supervisor list 121 in association with a supervisee, a request for disclosure approval that is made to the supervisor selected according to an order of priority.

Specifically, the disclosure requesting section 111 causes the disclosure approval screen 210 to be displayed on the supervisor terminal apparatus 20 of the supervisor (Step S102). The term “according to an order of priority” means that, for example, a request is transmitted to a highest-priority supervisor and that the request is transmitted to a second-highest-priority supervisor when there is no response from the highest-priority supervisor. When supervisors are prioritized without being treated in the same order, this makes it possible to give a higher priority to, for example, a supervisor who replies quickly, or a supervisor who knows a supervisee better. This results in being able to disclose vital data promptly and thus to perform an appropriate examination earlier.

FIG. 4 illustrates a flow of an operation performed by the supervisor terminal apparatus.

The disclosure approval section 201 of the supervisor terminal apparatus 20 receives, from the service server 10, the request for an approval for disclosure of vital data regarding the supervisee, and displays thereon the disclosure approval screen 210 (FIG. 1) (Step S201).

The disclosure approval screen 210 is a screen used to individually approve disclosure of pieces of vital data of different types (such as a body temperature, a heart rate, a breathing rate, and a blood pressure). The supervisor individually checks a check box 211 indicating a type of vital data that can be approved for disclosure (for example, a heart rate), and presses an OK button 212. Accordingly, the supervisor performs input on the disclosure approval section 201 to individually approve disclosure of pieces of vital data of different types. The disclosure approval section 201 accepts the disclosure approval input by the supervisor (Step S202).

The provision processing section 202 performs processing to provide the service server 10 with disclosure-approval-target vital data (a heart rate in this example) (Step S203). An example of processing to provide the service server 10 with vital data is described. For example, it is sufficient if the provision processing section 202 acquires a piece of disclosure-approval-target vital data (a heart rate) from the supervisee database 40 (the wearable device 60, a mobile device of the supervisee, or at least one storage apparatus in a network) in which pieces of vital data are accumulated, and supplies the piece of disclosure-approval-target vital data to the service server 10. Alternatively, the provision processing section 202 may report a disclosure-approval target (a heart rate) to the service server 10, and may cause the service server 10 to acquire vital data of the disclosure-approval target (a heart rate) from the supervisee database 40 (the wearable device 60, a mobile device of the supervisee, or at least one storage apparatus in a network). Alternatively, the provision processing section 202 may report a piece of disclosure-approval-target vital data (a heart rate) to the supervisee database 40 (the wearable device 60, a mobile device of the supervisee, or at least one storage apparatus in a network), and may cause the supervisee database 40 to transmit the piece of disclosure-approval-target vital data to the service server 10. The adoption of these methods results in synchronizing vital data in the supervisee database 40 with vital data in the service server 10.

The vital data acquiring section 112 of the service server 10 receives a piece of vital data (a heart rate) recorded in the supervisee database 40, and accumulates the pieces of vital data in the vital database 122 (Step S103). The service providing section 101 of the service server 10 causes a viewer to view vital data regarding the examination-target supervisee through the viewer terminal apparatus 30 (Step S104).

On the other hand, the approval information recording section 203 of the supervisor terminal apparatus 20 records the disclosure approval information 500 regarding disclosure approval (FIG. 1) in the history recording system 50 in a network (Step S204). The history recording system 50 in the network may be a blockchain, or may also be an undistributed storage apparatus (such as a server).

The disclosure approval information 500 includes date-and-time information 501, information 502 indicating a type of disclosure-target vital data (a heart rate), information 503 used to identify a supervisee (such as a name and an ID), information 504 used to identify a supervisor (such as a name and an ID), and information 505 used to identify a disclosure-destination service server 10 (such as a name and an ID). The date-and-time information 501 may correspond to a date and time at which a disclosure approval was accepted (Step S202), or may also correspond to a date and time at which processing to provide vital data was performed (Step S203).

It is difficult to falsify data recorded on a blockchain. In particular, recording of the disclosure approval information 500 on a blockchain that serves as the history recording system 50 makes it possible to record, in the form of tamper-proof traces, information (500) indicating when (501) and who (504) determined that a disclosure approval for which vital data (502) of whom (503) is to be given. With Web 3.0, there is essentially no need to register user information upon using services, which is different from Web 2.0. Thus, it is understood that, when there is a need to disclose vital data, timings at which management determination is performed are successively necessary. There may be a plurality of supervisors (such as a mother and a father) or there may be a change in supervisor (such as a change in a lawyer serving as a guardian). When it is necessary that supervisors perform management determination successively, recording of information regarding when and who determined that a disclosure approval for which vital data is to be given is especially useful in such a case.

### II. Second Embodiment

Hereinafter, descriptions and illustrations of a configuration and an operation that are similar to the configuration and operation described in the embodiment above are omitted, and a different configuration and a different operation are primarily described and illustrated.

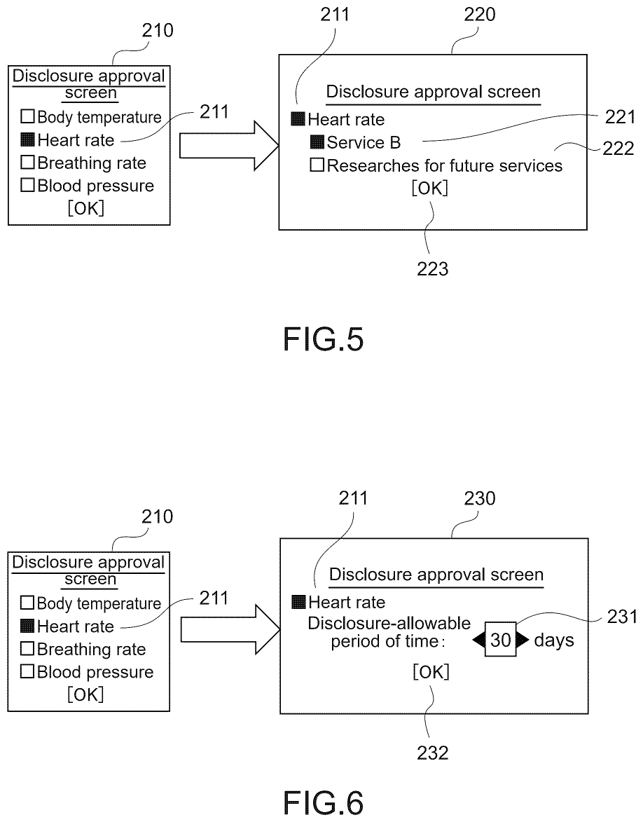

FIG. 5 illustrates an example of the disclosure approval screen according to a second embodiment.

With respect to the type of vital data (such as a heart rate 211) approved for disclosure on the disclosure approval screen 210 (Step S102), the disclosure requesting section 111 of the service server 10 causes another disclosure approval screen 220 to be displayed on the supervisor terminal apparatus 20 (Step S201). The disclosure approval screen 220 is a screen used to specify a purpose (that can also be referred to as application) for which vital data (a heart rate) approved for disclosure is allowed to be disclosed and to approve the disclosure. Examples of the purpose include a purpose 221 related to a current online-examination service provided by the service server 10, and a purpose 222 related to researches for feature services provided by the service server 10. A supervisor individually checks a check box 221 indicating a purpose (for example, an online-examination service) for which vital data (a heart rate) approved for disclosure is allowed to be disclosed, and presses an OK button 223. Accordingly, the supervisor inputs, to the disclosure approval section 201, a purpose for which vital data (a heart rate) approved for disclosure is allowed to be disclosed (Step S202). Note that, when there are pieces of vital data of different types as disclosure-approval targets, an individual disclosure approval screen 220 is displayed for each type of the piece of vital data, and a disclosure-allowable purpose can be individually specified for each type of the piece of vital data.

The provision processing section 202 performs processing to provide the service server 10 with disclosure-approval-target vital data (a heart rate in this example), and further the provision processing section 202 reports a disclosure-allowable purpose (for example, an online-examination service) to the service server 10 (Step S203).

The vital data acquiring section 112 of the service server 10 receives vital data (a heart rate) recorded in the supervisee database 40, and a report about a disclosure-allowable purpose (an online-examination service), and performs accumulation in the vital database 122 (Step S103). The service providing section 101 of the service server 10 causes a viewer to view vital data regarding an examination-target supervisee through the viewer terminal apparatus 30, and reports, to the viewer, a disclosure-allowable purpose (an online-examination service) (Step S104).

With respect to a disclosure-allowable purpose (an online-examination service), the service providing section 101 of the service server 10 can provide, in the future, another viewer with vital data currently accumulated in the vital database 122. On the other hand, for a purpose (for example, researches for future services) other than the disclosure-allowable purpose, the service providing section 101 does not externally provide the vital data currently accumulated in the vital database 122.

On the other hand, the disclosure approval information 500 that the approval information recording section 203 of the supervisor terminal apparatus 20 records (Step S204) in the history recording system 50 further includes information indicating a disclosure-allowable purpose.

The present embodiment enables a supervisor to determine a disclosure-allowable purpose for each type of vital data. Information regarding when and who determined that a disclosure approval for which vital data and for which purpose is to be given can be recorded.

### III. Third Embodiment

FIG. 6 illustrates an example of the disclosure approval screen according to a third embodiment.

With respect to the type of vital data (such as the heart rate 211) approved for disclosure on the disclosure approval screen 210 (Step S102), the disclosure requesting section 111 of the service server 10 causes another disclosure approval screen 230 to be displayed on the supervisor terminal apparatus 20 (Step S201). The disclosure approval screen 230 is a screen used to specify a period of time for which vital data (a heart rate) approved for disclosure is allowed to be disclosed and to approve for the disclosure. The period of time is, for example, the number 231 of days. A supervisor inputs a numerical value indicating a period of time (for example, 30 days) for which vital data (a heart rate) approved for disclosure is allowed to be disclosed, and presses an OK button 232. Accordingly, the supervisor inputs, to the disclosure approval section 201, a period of time for which vital data (a heart rate) approved for disclosure is allowed to be disclosed (Step S202). Note that, when there are pieces of vital data of different types as disclosure-approval targets, an individual disclosure approval screen 230 is displayed for each type of the piece of vital data, and a disclosure-allowable period of time can be individually specified for each type of the piece of vital data.

The provision processing section 202 performs processing to provide the service server 10 with disclosure-approval-target vital data (a heart rate in this example), and further the provision processing section 202 reports a disclosure-allowable period of time (for example, 30 days) to the service server 10 (Step S203).

The vital data acquiring section 112 of the service server 10 receives vital data (a heart rate) recorded in the supervisee database 40, and a report about a disclosure-allowable period of time (30 days), and performs accumulation in the vital database 122 (Step S103). The service providing section 101 of the service server 10 causes a viewer to view vital data regarding an examination-target supervisee through the viewer terminal apparatus 30, and reports, to the viewer, a disclosure-allowable period of time (30 days) (Step S104).

With respect to a disclosure-allowable period of time (30 days), the service providing section 101 of the service server 10 can provide, in the future, another viewer with vital data currently accumulated in the vital database 122. On the other hand, for a period of time (after the elapse of 30 days) other than the disclosure-allowable period of time, the service providing section 101 does not externally provide the vital data currently accumulated in the vital database 122. The service providing section 101 may delete vital data from the vital database 122 after the elapse of the disclosure-allowable period of time (30 days).

On the other hand, the disclosure approval information 500 that the approval information recording section 203 of the supervisor terminal apparatus 20 records (Step S204) in the history recording system 50 further includes information indicating a disclosure-allowable period of time.

The present embodiment enables a supervisor to determine a disclosure-allowable period of time for each type of vital data. Information regarding when and who determined that a disclosure approval for which vital data and for which period of time is to be given can be recorded.

### IV. Fourth Embodiment

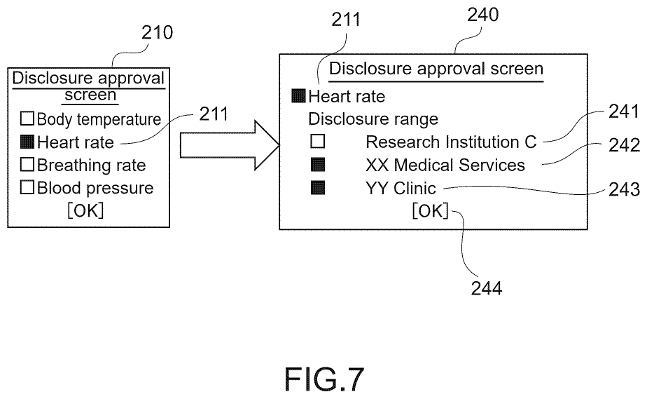

FIG. 7 illustrates an example of the disclosure approval screen according to a fourth embodiment.

With respect to the type of vital data (such as the heart rate 211) approved for disclosure on the disclosure approval screen 210 (Step S102), the disclosure requesting section 111 of the service server 10 causes another disclosure approval screen 240 to be displayed on the supervisor terminal apparatus 20 (Step S201). The disclosure approval screen 240 is a screen used to specify a user (that can also be referred to as a disclosure range) to which vital data (a heart rate) approved for disclosure is allowed to be disclosed and to approve the disclosure. Examples of the user include a specific research institution, a specific day-care-service company, and a specific clinic. Names of the users (for example, C Research Institution 241, XX Medical Services 242, and YY Clinic 243) are displayed on the disclosure approval screen 240. A supervisor individually checks check boxes 242 and 243 respectively indicating users (for example, XX Medical Services and YY Clinic) to which vital data (a heart rate) approved for disclosure is allowed to be disclosed, and presses an OK button 244. Accordingly, the supervisor inputs, to the disclosure approval section 201, a user to which vital data (a heart rate) approved for disclosure is allowed to be disclosed (Step S202). Note that, when there are pieces of vital data of different types as disclosure-approval targets, an individual disclosure approval screen 240 is displayed for each type of the piece of vital data, and a disclosure-allowable user can be individually specified for each type of the piece of vital data.

The provision processing section 202 performs processing to provide the service server 10 with disclosure-approval-target vital data (a heart rate in this example), and further the provision processing section 202 reports disclosure-allowable users (for example, XX Medical Services and YY Clinic) to the service server 10 (Step S203).

The vital data acquiring section 112 of the service server 10 receives vital data (a heart rate) recorded in the supervisee database 40, and a report about disclosure-allowable users (XX Medical Services and YY Clinic), and performs accumulation in the vital database 122 (Step S103). The service providing section 101 of the service server 10 causes a viewer to view vital data regarding an examination-target supervisee through the viewer terminal apparatus 30 (an online-examination service) (Step S104).

The service providing section 101 of the service server 10 can provide, in the future, disclosure-allowable users (XX Medical Services and YY Clinic) with vital data currently accumulated in the vital database 122. On the other hand, the service providing section 101 does not provide a user (for example, C Research Institution) other than the disclosure-allowable users with the vital data currently accumulated in the vital database 122.

On the other hand, the disclosure approval information 500 that the approval information recording section 203 of the supervisor terminal apparatus 20 records (Step S204) in the history recording system 50 further includes information indicating a disclosure-allowable user.

The present embodiment enables a supervisor to determine a disclosure-allowable user for each type of vital data. Information regarding when and who determined that a disclosure approval for which vital data and for which user is to be given can be recorded.

### V. Fifth Embodiment

When the service server 10 records a plurality of supervisors in the supervisor list 121, the service server 10 may record the supervisors in association with priorities for each of pieces of vital data of different types. In this case, the disclosure requesting section 111 of the service server 10 may transmit a request for disclosure approval that is made to a supervisor selected according to an order of priority set for the type of disclosure-approval-target vital data, and may cause the disclosure approval screens 210 to be displayed on the supervisor terminal apparatuses 20 of the different supervisors for the respective types of pieces of vital data (Step S102). When supervisors are prioritized for each of pieces of vital data of different types without being treated in the same order with respect to the pieces of vital data of different types, this makes it possible to give a higher priority to a supervisor who knows a supervisee's piece of vital data of a specific type better. For example, a mother is given a higher priority for a heart rate, and a father is given a higher priority for a body temperature. This results in being able to disclose pieces of vital data of different types appropriately and thus to perform an appropriate examination.

The present embodiment enables different supervisors to respectively determine disclosure approvals for respective types of pieces of vital data.

### VI. Others

As a modification, the disclosure approval information 500 may further include information indicating a user allowed to read, from the history recording system 50, the disclosure approval information 500 recorded in the history recording system 50.

The embodiments and modification described above may be combined discretionarily as necessary. For example, all of the purpose (the second embodiment), the period of time (the third embodiment), and the user (the fourth embodiment) can be specified for each attribute for vital data approved for disclosure.

The present disclosure may also include the following configurations.
(1) a Terminal Apparatus, Including:
   a disclosure approval section
      that receives, from a service server, a request for an approval for disclosure of data regarding a first user, the data being accumulated in a database, and
      that accepts the disclosure approval input by a second user who is different from the first user;
   a provision processing section that performs processing to provide the service server with the data, which is a target for the disclosure approval; and
   an approval information recording section that records disclosure approval information regarding the disclosure approval in a history recording system in a network.
(2) The terminal apparatus according to (1), in which
   the disclosure approval information includes date-and-time information, information used to identify the first user, information used to identify the second user, and information used to identify the service server.
(3) The terminal apparatus according to (1) or (2), in which
   the disclosure approval section individually approves disclosure of the pieces of data of different types, and
   the disclosure approval information further includes information indicating the type of the piece of data.
(4) The terminal apparatus according to any one of (1) to (3), in which
   the disclosure approval section specifies a purpose for which the disclosure-approval-target data is allowed to be disclosed, and
   the disclosure approval information further includes information indicating the purpose.
(5) The terminal apparatus according to any one of (1) to (4), in which
   the disclosure approval section specifies a period of time for which the disclosure-approval-target data is allowed to be disclosed, and
   the disclosure approval information further includes information indicating the period of time.
(6) The terminal apparatus according to any one of (1) to (5), in which
   the disclosure approval section specifies a user to which the disclosure-approval-target data is allowed to be disclosed, and
   the disclosure approval information further includes information indicating the user.
(7) The terminal apparatus according to any one of (1) to (6), in which
   the provision processing section acquires the disclosure-approval-target data from the database, and supplies the acquired data to the service server.
(8) The terminal apparatus according to any one of (1) to (6), in which
   the provision processing section reports the target for the disclosure approval to the service server, and causes the service server to acquire the disclosure-approval-target data from the database.
(9) The terminal apparatus according to any one of (1) to (8), in which
   the database is provided to at least one of
      a wearable device worn by the first user,
      a mobile device that communicates with the wearable device,
      the terminal apparatus that communicates with the wearable device or the mobile device, or
      at least one storage apparatus, in a network, that communicates with the wearable device, the mobile device, or the terminal apparatus.
(10) The terminal apparatus according to any one of (1) to (9), in which
   the history recording system in the network is a blockchain.
(11) The terminal apparatus according to any one of (1) to (10), in which
   the data regarding the first user is vital data of the first user.
(12) The terminal apparatus according to any one of (1) to (11), in which
   the second user is a supervisor of the first user.
(13) The terminal apparatus according to any one of (1) to (12), in which
   the service server records the second user or a plurality of the second users in association with the first user, and
   when the service server records the plurality of the second users,
      the service server records the plurality of the second users in association with priorities, and transmits the request for the disclosure approval, the request being made to the second user selected according to an order of priority.
(14) The terminal apparatus according to any one of (1) to (13), in which
   when the service server records a plurality of the second users,
      the service server records the plurality of the second users in association with priorities for each of the pieces of data of different types, and transmits the request for the disclosure approval, the request being made to the second user selected according to an order of priority set for the type of the piece of disclosure-approval-target data,
   the disclosure approval section individually approves disclosure of the pieces of data of the different types, and
   the disclosure approval information further includes information indicating the type of the piece of data.
(15) An information processing program that causes a control circuit of a terminal apparatus to operate as a disclosure approval section, a provision processing section, and an approval information recording section,
   the disclosure approval section receiving, from a service server, a request for an approval for disclosure of data regarding a first user, the data being accumulated in a database, the disclosure approval section accepting the disclosure approval input by a second user who is different from the first user,
   the provision processing section performing processing to provide the service server with the data, which is a target for the disclosure approval,
   the approval information recording section recording disclosure approval information regarding the disclosure approval in a history recording system in a network.
(16) An information processing system, including:
   a service server; and
   a terminal apparatus including
      a disclosure approval section
         that receives, from the service server, a request for an approval for disclosure of data regarding a first user, the data being accumulated in a database, and
         that accepts the disclosure approval input by a second user who is different from the first user,
      a provision processing section that performs processing to provide the service server with the data, which is a target for the disclosure approval, and
      an approval information recording section that records disclosure approval information regarding the disclosure approval in a history recording system in a network.
(17) A non-transitory computer-readable recording medium that records therein an information processing program that causes a control circuit of a terminal apparatus to operate as a disclosure approval section, a provision processing section, and an approval information recording section,
   the disclosure approval section receiving, from a service server, a request for an approval for disclosure of data regarding a first user, the data being accumulated in a database, the disclosure approval section accepting the disclosure approval input by a second user who is different from the first user,
   the provision processing section performing processing to provide the service server with the data, which is a target for the disclosure approval,
   the approval information recording section recording disclosure approval information regarding the disclosure approval in a history recording system in a network.

The embodiments and the modifications of the present technology have been described above. Of course the present technology is not limited to the embodiments described above, and various modifications may be made thereto without departing from the scope of the present technology.

### REFERENCE SIGNS LIST

1 information processing system
10 service server
101 service providing section
111 disclosure requesting section
112 vital data acquiring section
121 supervisor list
122 vital database
20 supervisor terminal apparatus
201 disclosure approval section
202 provision processing section
203 approval information recording section
30 viewer terminal apparatus
40 supervisor database
50 history recording system
500 disclosure approval information
60 wearable device

1. A terminal apparatus, comprising:
a disclosure approval section
that receives, from a service server, a request for an approval for disclosure of data regarding a first user, the data being accumulated in a database, and
that accepts the disclosure approval input by a second user who is different from the first user;
a provision processing section that performs processing to provide the service server with the data, which is a target for the disclosure approval; and
an approval information recording section that records disclosure approval information regarding the disclosure approval in a history recording system in a network.

2. The terminal apparatus according to claim 1, wherein
the disclosure approval information includes date-and-time information, information used to identify the first user, information used to identify the second user, and information used to identify the service server.

3. The terminal apparatus according to claim 1, wherein
the disclosure approval section individually approves disclosure of the pieces of data of different types, and
the disclosure approval information further includes information indicating the type of the piece of data.

4. The terminal apparatus according to claim 1, wherein
the disclosure approval section specifies a purpose for which the disclosure-approval-target data is allowed to be disclosed, and
the disclosure approval information further includes information indicating the purpose.

5. The terminal apparatus according to claim 1, wherein
the disclosure approval section specifies a period of time for which the disclosure-approval-target data is allowed to be disclosed, and
the disclosure approval information further includes information indicating the period of time.

6. The terminal apparatus according to claim 1, wherein
the disclosure approval section specifies a user to which the disclosure-approval-target data is allowed to be disclosed, and
the disclosure approval information further includes information indicating the user.

7. The terminal apparatus according to claim 1, wherein
the provision processing section acquires the disclosure-approval-target data from the database, and supplies the acquired data to the service server.

8. The terminal apparatus according to claim 1, wherein
the provision processing section reports the target for the disclosure approval to the service server, and causes the service server to acquire the disclosure-approval-target data from the database.

9. The terminal apparatus according to claim 1, wherein
the database is provided to at least one of
a wearable device worn by the first user,
a mobile device that communicates with the wearable device,
the terminal apparatus that communicates with the wearable device or the mobile device, or
at least one storage apparatus, in a network, that communicates with the wearable device, the mobile device, or the terminal apparatus.

10. The terminal apparatus according to claim 1, wherein
the history recording system in the network is a blockchain.

11. The terminal apparatus according to claim 1, wherein
the data regarding the first user is vital data of the first user.

12. The terminal apparatus according to claim 1, wherein
the second user is a supervisor of the first user.

13. The terminal apparatus according to claim 1, wherein
the service server records the second user or a plurality of the second users in association with the first user, and
when the service server records the plurality of the second users,
the service server records the plurality of the second users in association with priorities, and transmits the request for the disclosure approval, the request being made to the second user selected according to an order of priority.

14. The terminal apparatus according to claim 1, wherein
when the service server records a plurality of the second users,
the service server records the plurality of the second users in association with priorities for each of the pieces of data of different types, and transmits the request for the disclosure approval, the request being made to the second user selected according to an order of priority set for the type of the piece of disclosure-approval-target data,
the disclosure approval section individually approves disclosure of the pieces of data of the different types, and
the disclosure approval information further includes information indicating the type of the piece of data.

15. An information processing program that causes a control circuit of a terminal apparatus to operate as a disclosure approval section, a provision processing section, and an approval information recording section,
the disclosure approval section receiving, from a service server, a request for an approval for disclosure of data regarding a first user, the data being accumulated in a database, the disclosure approval section accepting the disclosure approval input by a second user who is different from the first user,
the provision processing section performing processing to provide the service server with the data, which is a target for the disclosure approval,
the approval information recording section recording disclosure approval information regarding the disclosure approval in a history recording system in a network.

16. An information processing system, comprising:
a service server; and
a terminal apparatus including
a disclosure approval section
that receives, from the service server, a request for an approval for disclosure of data regarding a first user, the data being accumulated in a database, and
that accepts the disclosure approval input by a second user who is different from the first user,
a provision processing section that performs processing to provide the service server with the data, which is a target for the disclosure approval, and
an approval information recording section that records disclosure approval information regarding the disclosure approval in a history recording system in a network.
